(19) United States
(12) Patent Application Publication
(10) Pub. No.: US 2020/0397749 A1
(43) Pub. Date: Dec. 24, 2020

(54) COMPOSITIONS AND METHODS FOR TREATING ACUTE CANNABINOID OVERDOSE WITH A CANNABINOID RECEPTOR ANTAGONIST

(71) Applicant: Opiant Pharmaceuticals, Inc., Santa Monica, CA (US)

(72) Inventor: Phil SKOLNICK, Potomac, MD (US)

(21) Appl. No.: 16/904,403

(22) Filed: Jun. 17, 2020

Related U.S. Application Data
(63) Continuation-in-part of application No. 16/704,699, filed on Dec. 5, 2019.
(60) Provisional application No. 63/037,351, filed on Jun. 10, 2020.
(60) Provisional application No. 62/862,832, filed on Jun. 18, 2019.
(60) Provisional application No. 62/862,830, filed on Jun. 18, 2019.

(51) Int. Cl. A61K 31/397; A61K 9/107; A61K 47/18; A61K 47/44; A61K 47/12; A61K 47/26; A61K 9/00; A61P 39/02

(52) U.S. Cl. CPC A61K 31/397; A61K 9/107; A61K 47/18; A61P 39/02; A61K 47/12; A61K 47/26; A61K 9/0019; A61K 47/44

(57) ABSTRACT

Provided are formulations and methods for treating, reversing, or reducing acute cannabinoid overdose, cannabinoid hyperemesis syndrome, or one or more symptoms thereof, comprising parenterally administering a CB1 antagonist in an amount sufficient to reverse the acute cannabinoid overdose, cannabinoid hyperemesis syndrome, or symptom(s) thereof.

This application claims the benefit of priority of U.S. Provisional Application No. 63/037,351, filed Jun. 10, 2020, U.S. Provisional Application No. 62/862,832, filed Jun. 18, 2019, U.S. Provisional Application No. 62/862,830, filed Jun. 18, 2019, and U.S. application Ser. No. 16/704,699, filed Dec. 5, 2019, the disclosures of which are hereby incorporated by reference as if written herein in their entireties.

Acute cannabinoid overdose (ACO) [also referred to as acute cannabinoid intoxication/poisoning] is most often linked to the consumption of “edibles” (sold as brownies, cookies, and candies) containing large quantities of D9-tetrahydrocannabinol (THC). Synthetic cannabinoids (SCs), initially developed as research tools following the identification of cannabinoid (CB) receptors, also pose a significant risk of producing an ACO. Multiple SCs have been reported to be more potent and efficacious than THC. Symptoms of ACO produced by both edibles and SCs include panic and anxiety, feelings of paranoia, agitation, visual and auditory hallucinations, and nausea. There are no FDA approved medications to treat ACO. Treatment is supportive and symptom driven, requiring emergency medical attention and in some instances, hospitalization.

Cannabinoid use can also produce a syndrome which includes incapacitating, cyclic bouts of nausea and vomiting. The severe nausea and vomiting that is associated with chronic cannabinoid use is commonly referred to as cannabinoid hyperemesis syndrome (CHS). CHS can be viewed as paradoxical, since a synthetic form of Δ⁹-tetrahydrocannabinol (THC), the principal psychoactive compound present in cannabis, has been approved by the U.S. FDA (and regulatory authorities in other countries) for the treatment of chemotherapy-induced nausea and vomiting.

Patients with CHS present to the emergency department (ED) with periodic episodes of intractable vomiting that are generally unresponsive to standard antiemetics such as ondansetron and promethazine. These patients can first exhibit severe anxiety and agitation, flushing, and sweating. Patients frequently also experience abdominal pain and severe nausea that can be triggered by the sight or smell of food. These symptoms are followed by a second phase consisting of severe, often incapacitating nausea and vomiting. During this period, patients often take very hot baths to relieve these symptoms; this can result in burns from immersion in scalding water. Resolution of nausea and vomiting may require 24-48 hours, during which time patients receive fluids and electrolyte replacement. Complete cessation of cannabinoid use during this period is critical for symptom relief. However, the interval from cessation of cannabis use to complete resolution of symptoms (e.g. abdominal pain) may be as along as 1-4 weeks. Patients treated for CHS in the emergency department often require admission to better manage both the emesis and the ensuing dehydration, electrolyte imbalance, and esophagitis that result from the frequent and severe vomiting. Authorities consider CHS distinct from acute cannabinoid overdose (also known as acute cannabinoid poisoning or intoxication) because of distinct symptoms and temporal patterning.

Following the identification of cannabinoid receptors in 1990, the pharmaceutical sector developed multiple, high affinity CB-1 receptor antagonists; a handful of these entered clinical trials. One of those molecules is drinabant (AVE-1625) was studied in Phase I and II studies.

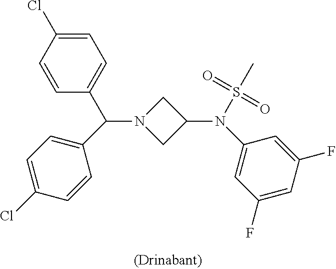 [SMILES: CS(=O)(=O)N(c1cc(F)cc(F)c1)C1CN(C(c2ccc(Cl)cc2)c2ccc(Cl)cc2)C1]

Oral doses of up to 240 mg were administered for up to 3 weeks in single- or repeated-dose Phase I studies, and 60 mg administered for up to 24 weeks in Phase II studies. Phase II studies were conducted in dyslipidemia, mild-to-moderate Alzheimer's disease, and in schizophrenia (as an add-on medication to treat cognitive impairment). Drinabant was safe and well tolerated in these studies, with a preponderance of GI related (nausea, vomiting, and diarrhea) adverse events. However, the slow onset of oral drinabant (Tmax>3 h) makes it impractical to administer in ACO.

There is a significant, unmet need for methods for treating, reversing, or reducing the symptoms of acute cannabinoid overdose, and for treating, reversing, or reducing the symptoms of cannabinoid hyperemesis syndrome. The present disclosure fulfills these and other needs, as evident in reference to the following disclosure.

### SUMMARY

Provided is a parenteral fluid concentrate comprising:

an amount of drinabant or a salt or polymorph thereof effective to reverse acute cannabinoid overdose, or one or more symptoms thereof in a subject in need thereof; and

at least one agent that acts as a non-ionic solubilizer and/or emulsifying agent.

Also provided is a parenteral fluid comprising a parenteral fluid concentrate described herein diluted with a pharmaceutically acceptable aqueous carrier.

Also provided is a method of treating, reversing, or reducing one or more symptoms of acute cannabinoid overdose comprising parenterally administering a parenteral fluid described herein to a subject in need thereof.

Also provided is a method of treating, reversing, or reducing cannabinoid hyperemesis syndrome or one or more symptoms thereof comprising parenterally administering a parenteral fluid described herein to a subject in need thereof.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

### Definitions

When ranges of values are disclosed, and the notation “from n₁ . . . to n₂” or “between n₁ . . . and n₂” is used, where n₁ and n₂ are numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 mL (milliliters),” which is intended to include 1 mL, 3 mL, and everything in between to any number of significant figures (e.g., 1.255 mL, 2.1 mL, 2.9999 mL, etc.).

As used herein, the term “about” is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

As used herein, reversal of symptom(s) of acute cannabinoid overdose is “apparent” when, in the judgment of a trained healthcare giver (e.g., physician, nurse practitioner, nurse, paramedic, or emergency medical technician), the symptom(s) have abated to a noticeable degree. Such a caregiver may use any appropriate measure to quantify the reversal of symptom(s), e.g., a visual analog scale for self-reporting, a heart rate monitor for tachycardia, etc. “Apparent” reversal of symptom(s) includes, but need not extend to, complete reversal.

As used herein, the term “cannabinoid” is synonymous with “cannabinoid receptor agonist” and refers to a compound which binds to and activates a cannabinoid receptor. The term includes both natural and synthetic compounds.

As used herein, the term “synthetic cannabinoid” (“SC”) means a non-naturally-occurring cannabinoid. While not synthetic analogues of THC and other naturally occurring cannabinoids, SCs share many common features with THC. Most are lipid-soluble, non-polar, small molecules (usually about 20-26 carbon atoms) that are fairly volatile, and often have a side-chain of 5-9 saturated carbon atoms which is associated with psychotropic activity from binding CB1 receptors. There are at least five major structural categories for synthetic cannabinoids: classical cannabinoids, non-classical cannabinoids, hybrid cannabinoids, aminoalkylindoles (and their analogues), and eicosanoids. Classical cannabinoids are analogs of THC that are based on a dibenzopyran ring; examples include and Compounds such as HU-210 (the (-)-1,1-dimethylheptyl analog of 11-hydroxy-Δ8-tetrahydrocannabinol (HU-210)) are synthetic analogs of THC. Other chemical classes include aminoakylindoles including naphthoylindoles such as 1-pentyl-3-(1-naphthoyl)indole (JWH-018), phenylacetylindoles such as 1-pentyl-3-(2-methoxyphenylacetyl)indole (JWH-250), and benzoylindoles such as 1-[(N-methylpiperidin-2-yl)methyl]-3-(2-iodobenzoyl)indole (AM-2233); they are the most common SCs found in SC blends due to relative ease of synthesis. Other compounds structurally similar to aminoalkylindoles include naphthoylpyrroles, naphthylmethylindenes, phenylacetylindoles/benzoylindoles, tetramethylcyclopropylindoles, adamantoylindoles, indazole carboxamides, indolecarboxylates, and quinolinyl esters. Eicosanoid SCs are analogs of endocannabinoids such as anandamide.

As used herein, the term “cannabinoid receptor antagonist” refers to a compound which binds to and blocks or dampens the normal biological function of the receptor and its signaling, especially in the presence of an agonist or partial agonist. The term includes cannabinoid receptor antagonists that are selective or nonselective for the CB1 receptor subtype, i.e., a “CB1 antagonist.”

As used here, the term “cannabinoid hyperemesis syndrome” or “CHS” refers to the severe nausea and vomiting that is associated with chronic cannabinoid use. Authorities consider CHS distinct from acute cannabinoid overdose (also known as acute cannabinoid poisoning or intoxication) because of distinct symptoms and temporal patterning.

As used herein, the term “Cₘₐₓ” refers to the maximum observed plasma concentration.

As used herein, the term “intramuscular (IM)” means into a muscle. Suitable muscles, if of sufficient mass, include the deltoid (upper arm), the thigh (esp. the anterolateral aspect of the thigh; particularly useful if via an autoinjector), the gluteus maximus (typically only adults and children >3 years old), and hip. The IM injection may be via a classical syringe or an autoinjector device.

As used herein, the term “intravenous (IV)” means delivered as a liquid into a vein of a patient. Intravenous administration can be by injection (in a relatively small volume and at relatively high concentration) by injection via a syringe or into a previously-inserted IV catheter, or by intravenous infusion (“IVN,” in a relatively larger and more dilute volume). IV administration, particularly injection, can be done in one or more pushes.

The terms “non-ionic solubilizer” and/or “emulsifying agent” and/or “solubilizing agent” are generally interchangeable as used herein, and include agents that result in formation of a micellar solution or a true solution of the agent being solubilized and a typically immiscible partner (for example, drinabant, which has a high logP, and water, which has a negative logP). Solubilizing agents include cationic and nonionic surfactants, and in certain circumstances may also act as absorption or permeation enhancers. One example of a solubilizing agent is Kolliphor HS 15/Solutol HS 15 (e.g., macrogol 15 hydroxystearate, CAS No. 70142-34-6 or 61909-81-7, polyoxyl 15 hydroxystearate, polyglycol mono- and di-esters of 12-hydroxystearic acid with about 30% polyethylene glycol).

As used herein, the terms “overdose,” “intoxication,” and “poisoning” are synonymous and may be used interchangeably, and refer to the condition of having taken into the body of a subject, e.g. by inhalation or ingestion, an excess of a physiologically active and and/or psychoactive substance, such that the normal functioning of the body or one of its functions or parts is perturbed and the subject is at risk of harm.

As used herein, the term “parenteral” means administered by means other than oral, nasal (i.e., bypassing mucous membranes) or rectal intake, particularly intravenously or by injection elsewhere, e.g., intramuscular or subcutaneous injection.

As used herein, the term “push” in the context of an intravenous (IV) push is the rapid administration of a small volume of medication into a patient's vein, typically via a previously inserted IV catheter. Multiple pushes make be used to comprise a single IV dose.

As used herein, the term “subcutaneous” means “under the skin,” i.e., administered into the subcutis, the layer of skin directly below the dermis and epidermis (collectively referred to as the cutis), above muscle.

As used herein, a “symptom” of intoxication or overdose is a physical or mental feature that is regarded as is a departure from normal function or feeling. Common symptoms of cannabinoid intoxication or overdose include dry mouth, increased appetite, nystagmus, slurred speech, and conjunctival injection (red eye), as well as generalized psychomotor impairment, including impaired attention, reduced alertness (drowsiness), impaired concentration, slowed reaction time, impaired short-term memory, impaired executive functioning, and confusion. More serious symptoms may include postural/orthostatic hypotension, hypertension, tachycardia, nausea, delirium, agitation, anxiety, panic attacks, myoclonic jerking, sedation, paranoia, and hallucination. Severe effects may include seizures, hyperthermia, rhabdomyolysis, renal failure, angina, and myocardial infarction. Symptoms may present differently in children, and are known in the art. Symptoms of cannabinoid intoxication or overdose may be divided into cardiovascular symptom(s), neuropsychiatric symptom(s), and gastrointestinal symptom(s), with some overlap (e.g., a panic attack has both physical and neuropsychiatric components).

As used herein, the term “in need of treatment” and the term “in need thereof” when referring to treatment are used interchangeably and refer to a judgment made by a health caregiver (e.g. physician, nurse, nurse practitioner, that a patient will benefit from treatment.

As used herein, the term “subject” is intended to be synonymous with “patient,” and refers to any mammal (preferably human) who is intoxicated or overdosed with a cannabinoid.

### Description

Provided is a parenteral fluid concentrate comprising:

an amount of drinabant or a salt or polymorph thereof effective to i) treat, reverse, or reduce acute cannabinoid overdose or one or more symptoms thereof in a subject in need thereof, and/or ii) treat, reverse, or reduce cannabinoid hyperemesis syndrome (CHS) or one or more symptoms thereof in a subject in need thereof; and

at least one agent that acts as a non-ionic solubilizer and/or emulsifying agent.

In some embodiments, the amount of drinabant or a salt or polymorph thereof is effective to reverse acute cannabinoid overdose or one or more symptoms thereof in a subject in need thereof, and/or ii) reverse cannabinoid hyperemesis syndrome (CHS) or one or more symptoms thereof in a subject in need thereof. In some embodiments, the amount of drinabant or a salt or polymorph thereof is effective to reverse acute cannabinoid overdose or one or more symptoms thereof in a subject in need thereof. In some embodiments, the amount of drinabant or a salt or polymorph thereof is effective to reverse cannabinoid hyperemesis syndrome (CHS) or one or more symptoms thereof in a subject in need thereof.

In some embodiments, the at least one agent that acts as a non-ionic solubilizer and/or emulsifying agent is chosen from polyoxyethylene (20) sorbitan monooleate, polyoxyl 40 hydrogenated castor oil, polysorbate 80; macrogol 15 hydroxystearate, arid polyoxamers. In some embodiments, the at least one agent that acts as a non-ionic solubilizer and/or emulsifying agent is chosen from polysorbate 80 and macrogol 15 hydroxystearate. In some embodiments, the at least one agent that acts as a non-ionic solubilizer and/or emulsifying agent is macrogol 15 hydroxystearate.

In some embodiments, the concentrate further comprises at least one hydrophilic solvent. In some embodiments, the at least one hydrophilic solvent is chosen from ethanol, PEG 400, and propylene glycol.

In some embodiments, the concentrate comprises an amount of drinabant or a salt or polymorph thereof effective to reverse acute cannabinoid overdose or one or more symptoms thereof or reverse cannabinoid hyperemesis syndrome or one or more symptoms thereof in a subject and macrogol 15 hydroxystearate. In some embodiments, the concentrate consists essentially of an amount of drinabant or a salt or polymorph thereof effective to reverse acute cannabinoid overdose or one or more symptoms thereof or reverse cannabinoid hyperemesis syndrome or one or more symptoms thereof in a subject; and macrogol 15 hydroxystearate. In some embodiments, the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject; and macrogol 15 hydroxystearate.

In some embodiments, the concentrate comprises an amount of drinabant or a salt or polymorph thereof effective to reverse acute cannabinoid overdose or one or more symptoms thereof or reverse cannabinoid hyperemesis syndrome or one or more symptoms thereof in a subject, macrogol 15 hydroxystearate, and at least 20% ethanol. In some embodiments, the concentrate consists essentially of an amount of drinabant or a salt or polymorph thereof effective to reverse acute cannabinoid overdose or one or more symptoms thereof or reverse cannabinoid hyperemesis syndrome or one or more symptoms thereof in a subject, macrogol 15 hydroxystearate, and at least 20% ethanol. In some embodiments, the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject, macrogol 15 hydroxy⁻stearate, and at least 20% ethanol.

In some embodiments, the concentrate comprises an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 80% macrogol 15 hydroxystearate and 20% ethanol. In some embodiments, the concentrate consists essentially of an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 80% macrogol 15 hydroxystearate and 20% ethanol. In some embodiments, the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 80% macrogol 15 hydroxystearate and 20% ethanol.

In some embodiments, the concentrate comprises an amount of drinabant or a salt or polymorph thereof effective to reverse acute cannabinoid overdose or one or more symptoms thereof or reverse cannabinoid hyperemesis syndrome or one or more symptoms thereof in a subject, macrogol 15 hydroxystearate, and at least 30% propylene glycol. In some embodiments, the concentrate consists essentially of an amount of drinabant or a salt or polymorph thereof effective to reverse acute cannabinoid overdose or one or more symptoms thereof or reverse cannabinoid hyperemesis syndrome or one or more symptoms thereof in a subject, macrogol 1.5 hydroxystearate, and at least 30% propylene glycol. In some embodiments, the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject, macrogol 15 hydroxystearate, and at least 30% propylene glycol.

In some embodiments, the concentrate comprises an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 70% macrogol 15 hydroxystearate and 30% propylene glycol. In some embodiments, the concentrate consists essentially of an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 70% macrogol 15 hydroxystearate and 30% propylene glycol. In some embodiments, the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject, and a mixture of 70% macrogol 15 hydroxystearate and 30% propylene glycol.

In some embodiments, the concentrate comprises between about 2 and about 50 mg/g with drinabant or a salt or polymorph thereof. In some embodiments, the concentrate comprises between about 2 and about 40 mg/g with drinabant or a salt or polymorph thereof. In some embodiments, the concentrate comprises about 10 mg/g drinabant or a salt or polymorph thereof. In some embodiments, the concentrate comprises about 20 mg/g drinabant or a salt or polymorph thereof. In some embodiments, the concentrate comprises about 30 mg/g drinabant or a salt or polymorph thereof. In some embodiments, the concentrate comprises about 40 mg/g drinabant or a salt or polymorph thereof.

In some embodiments, the concentrate is stable for one week at room temperature.

In some embodiments, the concentrate further comprises at least one phospholipid. In some embodiments, the at least one phospholipid is chosen from lecithins of natural origin, phospholipids of natural origin, synthetic phospholipids, and a mixture thereof.

In some embodiments, the concentrate further comprises Miglyol 812 (medium chain triglycerides), a pharmaceutical oil, or a mixture thereof. In some embodiments, the pharmaceutical oil is chosen from soybean oil, olive oil, sesame oil, and hydrogenated vegetable oil.

In some embodiments, the concentrate further comprises at least one antioxidant. In some embodiments, the at least one antioxidant is chosen from ascorbic acid, monothioglycerol, cysteine HCl, and glutathione.

In some embodiments, the concentrate further comprises at least one isotonic agent. In some embodiments, the at least one isotonic agent is chosen from polyethylene glycol, glycerol, saline, and glucose.

Also provided is a parenteral fluid comprising a concentrate described herein diluted with a pharmaceutically acceptable aqueous carrier.

In some embodiments, the pharmaceutically acceptable aqueous carrier is chosen from sterile water, aqueous dextrose, saline, aqueous saline/dextrose, and aqueous glucose. In some embodiments, the pharmaceutically acceptable aqueous carrier is glucose 5% solution.

In some embodiments, the parenteral fluid comprises about 1 mg/mL drinabant or a salt or polymorph thereof.

In some embodiments, the parenteral fluid comprises about 1.5 mg/mL drinabant or a salt or polymorph thereof.

In some embodiments, the parenteralfluid comprises about 2 mg/mL drinabant or a salt or polymorph thereof.

In some embodiments, the parenteral fluid comprises about 2.5 mg/mL drinabant or a salt or polymorph thereof.

In some embodiments, the parenteral fluid comprises about 3 mg/mL drinabant or a salt or polymorph thereof.

In some embodiments, the parenteral fluid comprises about 3.5 mg/mL drinabant or a salt or polymorph thereof.

In some embodiments, the parenteral fluid comprises about 4 mg/mL drinabant or a salt or polymorph thereof.

In some embodiments, the parenteral fluid comprises about 4.5 mg/mL drinabant or a salt or polymorph thereof.

In some embodiments, the parenteral fluid comprises about 5 mg/mL drinabant or a salt or polymorph thereof.

In some embodiments, the concentrate or parenteral fluid comprises one or more antimicrobials (sometimes referred to as preservatives). The antimicrobial(s) may be present in a bacteriostatic, fungistatic, bacteriocidal, or fungicidal amount, and should avoid toxicity to the subject receiving the formulation. In some embodiments, the one or more preservative(s) is/are chosen from benzyl alcohol, phenol, p-hydroxybenzoic acid (PHBA), o-hydroxybenzoic acid (salicylic acid), one or more parabens or a mixture thereof (e.g., methylparaben, ethylparaben, n-propylparaben, isopropylparaben, butylparaben, and nipastat), a methylphenol (o-, m-, or p-hydroxytoluene) or a derivative thereof, benzalkonium chloride, benzethonium chloride, chlorobutanol, phenylmercuric nitrate, and thimerosal.

In some embodiments, the concentrate or parenteral fluid comprises one or more stabilizing agents (sometimes also referred to as preservatives or antioxidants). In some embodiments, the one or more stabilizing agent(s) is/are chosen from ethylenediaminetetraacetic acid or a salt thereof (ETDA, disodium EDTA), ascorbic acid, sodium bisulfite, and a sulfurous acid salt.

In some embodiments, the concentrate or parenteral fluid comprises one or more pH-adjusting agents, e.g. an acid (e.g., a strong such as HCl, or a weak acid such as citric acid) or a base (such as NaOH).

Also provided is a method of treating, reversing, or reducing one or more symptoms of acute cannabinoid overdose comprising parenterally administering a parenteral fluid described herein to a subject in need thereof.

In some embodiments, the symptom(s) of acute cannabinoid overdose is/are chosen from cardiovascular symptom(s), neuropsychiatric symptom(s), and gastrointestinal symptom(s).

In some embodiments, the cardiovascular symptom(s) is/are chosen from hypertension and tachycardia.

In some embodiments, the neuropsychiatric symptom(s) is/are chosen from agitation, confusion, drowsiness/lack of alertness, hallucinations, and feeling “high.”

In some embodiments, the gastrointestinal symptom(s) is/are chosen from nausea and vomiting.

In some embodiments, the onset of reversal of symptom(s) of acute cannabinoid overdose are apparent within 5-30 minutes following intravenous injection of drinabant, or a salt or polymorph thereof. In some embodiments, the onset of reversal of symptom(s) of acute cannabinoid overdose are apparent within 15-45 min following intramuscular administration of drinabant, or a salt or polymorph thereof.

In some embodiments, if no response is observed within 30-120 minutes of a first administration of drinabant, and the presence of acute cannabinoids is confirmed or strongly suspected, a second dose may be administered. In some embodiments, if no response is observed within 30-45 minutes of a first administration of drinabant, and the presence of cannabinoids is confirmed or strongly suspected, a second dose may be administered.

Also provided is a method of treating, reversing, or reducing cannabinoid hyperemesis syndrome or one or more symptoms thereof comprising parenterally administering a parenteral fluid described herein to a subject in need thereof.

In some embodiments, the symptom(s) of CHS is/are chosen from nausea, vomiting, and/or abdominal pain.

In some embodiments, the onset of reversal of symptom(s) of CHS are apparent within 5-30 minutes following intravenous injection of drinabant, or a salt or polymorph thereof. In some embodiments, the onset of reversal of symptom(s) of CHS are apparent within 15-45 min following intramuscular administration of drinabant, or a salt or polymorph thereof.

In some embodiments, the parenteral route of administration is chosen from among intravenous (IV), intramuscular (IM), and subcutaneous (SC).

In some embodiments, the parenteral route of administration is IV.

In some embodiments, the plasma concentrations achieved by parenteral administration of drinabant or salt or polymorph thereof is 200 to about 730 ng/ml.

In some embodiments, the administration of the drinabant, or a salt or polymorph thereof, provides a C. of 500 ng/ml.

In some embodiments, the amount of drinabant, or a salt or polymorph thereof, is between about 1 mg and about 60 mg per intravenous dose. In some embodiments, the amount of drinabant, or a salt or polymorph thereof is between about 30 and about 60 mg per intravenous dose.

In some embodiments, the IV dose is delivered by IV injection.

In some embodiments, the IV dose is delivered in a liquid volume of between about 1 and about 20 mL.

In some embodiments, the IV dose is delivered by IV infusion.

In some embodiments, the IV infusion is delivered in a liquid volume of between about 125 to about 500 mL.

In some embodiments, the IV infusion is delivered over a period of about 1 hour to about 2 hours. In some embodiments, the IV infusion is delivered at a rate of about 0.5 mL/min to about 2 mL/min

In some embodiments, the parenteral route of administration is IM or SC.

In some embodiments, the amount of drinabant, or a salt or polymorph thereof is between about 5 and about 60 mg per IM or SC dose. In some embodiments, the amount of drinabant, or a salt or polymorph thereof is between about 5 and about 30 mg per IM or SC dose .

In some embodiments, the intramuscular dose is delivered in a liquid volume of up to about 2.5 ml. In some embodiments, the intramuscular dose is delivered in a liquid volume of about 1 to about 2.5 ml.

In some embodiments, the SC dose is delivered in a liquid volume of up to about 1.5 ml. In some embodiments, the SC dose is delivered in a liquid volume of about 1 mL to about 1.5 ml.

Plasma concentrations of CB1 antagonists useful in treating, reversing, or reducing acute cannabinoid overdose or one or more symptoms thereof will vary based on several factors, including the identity of the antagonist. For example, plasma concentrations of drinabant useful in treating, reversing, or reducing acute cannabinoid overdose or one or more symptoms thereof range from about 200 ng/mL to about 730 ng/mL. Additionally, it is understood by those skilled in the art that the because drinabant has been characterized as a competitive CB1 receptor antagonist, effective (therapeutic) plasma concentrations are dependent upon the dose and type of cannabinoid (a SC, THC, or a combination thereof) responsible for the overdose. Both the onset and degree of symptom relief may vary, and some symptoms (e.g. cardiovascular symptoms such as tachycardia) may be more sensitive to reversal than others (e.g., alertness, as measured by a clinician's impression and/or assessment through a visual analog scale (VAS)). Onset of symptom relief should be apparent within about 5 min to about 30 min following intravenous administration and about 15 min to about 45 min following intramuscular or subcutaneous administration, respectively.

In order to achieve these plasma concentrations, intravenous doses of drinabant of between about 1 mg and about 150 mg, about 1 mg and about 100 mg, or between about 1 mg and about 60 mg, or between about 15 mg and about 60 mg, or between about 30 mg and about 60 mg, or between about 1 mg and about 30 mg, or between about 15 mg and about 30 mg, or between about 50 mg and about 100 mg may be administered. Intravenous doses can be injected in volumes of about 1 to about 20 mL (lower volumes are preferred in certain circumstances).

Alternatively, in order to achieve these plasma concentrations, intramuscular or subcutaneous doses of drinabant of between about 1 mg and about 150 mg, about 5 mg and about 100 mg, or between about 5 mg and about 60 mg, or between about 15 mg and about 60 mg, or between about 30 mg and about 60 mg, or between about 15 mg and about 30 mg, or between about 50 mg and about 100 mg, or between about 5 mg and about 50 mg, or between about 5 mg and about 30 mg. Intramuscular or subcutaneous doses can be injected in a volume of up to about 2.5 mL for IM and about 1.5 mL for SC. Intramuscular injections are typically into a deltoid or gluteal muscle.

### EXAMPLES

### Example 1

Table 1 below discloses several examples of compositions which can be formulated for parenteral administration (i.e., as liquid preparations) comprising an amount of drinabant or a salt or polymorph thereof effective to reverse acute cannabinoid overdose or one or more symptoms thereof or reverse cannabinoid hyperemesis syndrome or one or more symptoms thereof in a subject.

| | | Dose, | Vol, |
|---|---|---|---|
| Ex. | Mode | mg | mL |
| 1 | IV | 1 | 1 |
| 2 | IV | 1 | 2 |
| 3 | IV | 1 | 3 |
| 4 | IV | 1 | 5 |
| 5 | IV | 1 | 10 |
| 6 | IV | 5 | 1 |
| 7 | IV | 5 | 2 |
| 8 | IV | 5 | 3 |
| 9 | IV | 5 | 5 |
| 10 | IV | 5 | 10 |
| 11 | IV | 15 | 1 |
| 12 | IV | 15 | 2 |
| 13 | IV | 15 | 3 |
| 14 | IV | 15 | 5 |
| 15 | IV | 15 | 10 |
| 16 | IV | 30 | 1 |
| 17 | IV | 30 | 2 |
| 18 | IV | 30 | 3 |
| 19 | IV | 30 | 5 |
| 20 | IV | 30 | 10 |
| 21 | IV | 45 | 1 |
| 22 | IV | 45 | 2 |
| 23 | IV | 45 | 3 |
| 24 | IV | 45 | 5 |
| 25 | IV | 45 | 10 |
| 26 | IV | 60 | 1 |
| 27 | IV | 60 | 2 |
| 28 | IV | 60 | 3 |
| 29 | IV | 60 | 5 |
| 30 | IV | 60 | 10 |
| 31 | IV | 100 | 1 |
| 32 | IV | 100 | 2 |
| 33 | IV | 100 | 3 |
| 34 | IV | 100 | 5 |
| 35 | IV | 100 | 10 |
| 36 | IM | 5 | 1 |
| 37 | IM | 5 | 1.5 |
| 38 | IM | 5 | 2 |
| 39 | IM | 5 | 2.5 |
| 40 | IM | 15 | 1 |
| 41 | IM | 15 | 1.5 |
| 42 | IM | 15 | 2 |
| 43 | IM | 15 | 2.5 |
| 44 | IM | 30 | 1 |
| 45 | IM | 30 | 1.5 |
| 46 | IM | 30 | 2 |
| 47 | IM | 30 | 2.5 |
| 48 | IM | 45 | 1 |
| 49 | IM | 45 | 1.5 |
| 50 | IM | 45 | 2 |
| 51 | IM | 45 | 2.5 |
| 52 | IM | 60 | 1 |
| 53 | IM | 60 | 1.5 |
| 54 | IM | 60 | 2 |
| 55 | IM | 60 | 2.5 |
| 56 | IM | 100 | 1 |
| 57 | IM | 100 | 1.5 |
| 58 | IM | 100 | 2 |
| 59 | IM | 100 | 2.5 |
| 60 | SC | 5 | 0.5 |
| 61 | SC | 5 | 1 |
| 62 | SC | 5 | 1.5 |
| 63 | SC | 15 | 0.5 |
| 64 | SC | 15 | 1 |
| 65 | SC | 15 | 1.5 |
| 66 | SC | 30 | 0.5 |
| 67 | SC | 30 | 1 |
| 68 | SC | 30 | 1.5 |
| 69 | SC | 45 | 0.5 |
| 70 | SC | 45 | 1 |
| 71 | SC | 45 | 1.5 |
| 72 | SC | 60 | 0.5 |
| 73 | SC | 60 | 1 |
| 74 | SC | 60 | 1.5 |
| 75 | SC | 100 | 0.5 |
| 76 | SC | 100 | 1 |
| 77 | SC | 100 | 1.5 |

Table 2 below discloses several examples of compositions which can be formulated for parenteral administration as an IV infusion, comprising an amount of drinabant or a salt or polymorph thereof effective to reverse acute cannabinoid overdose or one or more symptoms thereof or reverse cannabinoid hyperemesis syndrome or one or more symptoms thereof in a subject. The drinabant is delivered, e.g., over the given time interval (period, in minutes) below.

TABLE 2

| | | Dose, | Vol., | Per., |
|---|---|---|---|---|
| Ex. | Mode | mg | mL | min |
| 78 | IVN | 5 | 50 | 30 |
| 79 | IVN | 5 | 100 | 30 |
| 80 | IVN | 5 | 125 | 30 |
| 81 | IVN | 15 | 50 | 30 |
| 82 | IVN | 15 | 100 | 30 |
| 83 | IVN | 15 | 125 | 30 |
| 84 | IVN | 30 | 125 | 60 |
| 85 | IVN | 30 | 125 | 90 |
| 86 | IVN | 30 | 125 | 120 |
| 87 | IVN | 30 | 250 | 60 |
| 88 | IVN | 30 | 250 | 90 |
| 89 | IVN | 30 | 250 | 120 |
| 90 | IVN | 45 | 250 | 60 |
| 91 | IVN | 45 | 250 | 90 |
| 92 | IVN | 45 | 250 | 120 |
| 93 | IVN | 45 | 500 | 60 |
| 94 | IVN | 45 | 500 | 90 |
| 95 | IVN | 45 | 500 | 120 |
| 96 | IVN | 60 | 250 | 60 |
| 97 | IVN | 60 | 250 | 90 |
| 98 | IVN | 60 | 250 | 120 |
| 99 | IVN | 60 | 500 | 60 |
| 100 | IVN | 60 | 500 | 90 |
| 101 | IVN | 60 | 500 | 120 |

### Example 2

### Assays and Protocols

Clinical Pharmacology Protocol for Cannabinoid Overdose

Pharmacokinetics. For determination of the concentration of plasma drinabant, venous blood may be collected in, e.g., heparinized polypropylene tubes (lithium heparin) of 4 mL. Blood samples may be taken at baseline and (by way of example only) 2.5, 5, 10, 15, 20, 30, and/or 45 min and 1, 1.5, 2, 3, 4, 5, 6, 12 and 24 h after oral administration of drinabant or matching placebo. After blood collection, the tubes are centrifuged within 30 min for 15 min at 2000 g at 4° C. Plasma samples may be stored at a temperature of −20° C.

Turbulent Flow Chromatography-Mass Spectrometry/Mass Spectrometry (TFC-MS/MS) is a validated method to analyze plasma drinabant concentrations. See, e.g., Zuurman, et al., 2010. Validation of this method included evaluation of selectivity for drinabant. In each run, standards (known amount of drinabant) man be included periodically (e.g., after every 10 samples). The limit of quantification has been reported in the art 0.2 ng/mL; the intra-assay coefficient of variation between 1.0 and 5.4%; the inter-assay coefficient of variation between 2.0 and 6.5%. Preferably, all blood samples are handled and analyzed according to GCP/GLP. Drinabant plasma pharmacokinetic parameters (including tₘₐₓ, Cₘₐₓ, AUC₀₋₂₄, AUC_{inf}f, t_{1/2}) may be determined using non-compartmental analysis from individual plasma concentration-time profiles.

Plasma concentrations of cannabinoids may be assessed by methods known in the art, e.g. as disclosed in Sørensen L K and Hasselstrøm J B, Sensitive Determination of Cannabinoids in Whole Blood by LC-MS-MS After Rapid Removal of Phospholipids by Filtration, J Anal Toxicol 2017 Jun. 1; 41(5):382-391.

Pharmacodynamics. Pharmacodynamic endpoints of the efficacy of various formulations and doses of cannabinoid antagonists such as drinabant in treating, reversing, or reducing cannabinoid overdose or one or more symptoms thereof can be measured using a variety of measures, including both objectively observable and subject-reported phenomena.

Objectively observable measures of symptoms of cannabinoid overdose include tachycardia and hypertension. Cannabis and other cannabinoids dose-dependently increase heart rate and blood pressure, and these symptoms can become life-threatening in certain subjects. Drinabant is expected to reduce tachycardia (i.e., reduce heart rate) and blood pressure in a subject intoxicated or overdosed with one or more cannabinoids.

Other objectively observable measures of symptoms of cannabinoid overdose include postural instability, measurable by methods known in the art. See, e.g., Browne J E and O'Hare N J, Review of the Different Methods for Assessing Standing Balance, Physiotherapy 2001 87(9):489-495. Cannabinoids tend to increase postural instability, and a cannabinoid antagonist such as drinabant would be expected to reverse this effect. Drinabant is expected to reduce postural instability in a subject intoxicated or overdosed with one or more cannabinoids.

Two of the most frequently used subject-reported scales are visual analogue scales (VASs) in clinical (pharmacologic) research to measure subjective effects: VAS Bond and Lader (alertness, calmness and mood) and VAS Bowdle (psychedelic effects). See, e.g., Kleinloog D et al., Profiling the subjective effects of Δ⁹-tetrahydrocannabinol using visual analogue scales, Int J Methods Psychiatr Res, 2014 June; 23(2):245-56. Three separate clusters may be monitored that describe the spectrum of subjective effects of cannabinoids, including the perception VAS (“time”, “thoughts” and “high”), the relaxation VAS (“drowsy”, “muzzy”, “mentally slow” and “dreamy”) and dysphoria VAS (“voices”, “meaning” and “suspicious”). The effects of cannabinoids on these VASs or any subsets thereof may be measured, and the effects of cannabinoid antagonists measured as well by the decline in these measures after administering an amount of a cannabinoid antagonist, such as drinabant, therapeutically effective to reverse cannabinoid intoxication. Drinabant is expected to reverse measures of cannabinoid intoxication and overdose.

Solubility Protocol

Drinabant may be added to a fixed volume of aqueous solution with and without various amounts of water soluble carriers such as Solutol HS 15 in screw capped bottles. Samples are shaken (alternatively, stirred) for a length of time (e.g., 48 hours) at room temperature, pH optionally adjusted, and any suspensions filtered through, e.g., a Whatman filter paper no 1. Filtered solutions are then analyzed for drinabant concentration using an appropriate method such as UV/visible spectrophotometry at an appropriate wavelength (nm) or by HPLC. It is expected that at low concentrations of solubilizing agent (e.g., 1, 5, or 10%), improvement in solubility will increase linearly, but that at higher concentrations this trend may deviate.

### Example 3

Two different categories of excipients, described in the literature as injectable intravenously, were tested for the solubility of drinabant:

1. Amphiphilic surfactants with hydrophilic character (HLB >10)

2. Hydrophilic solvents

The chemical composition of the selected excipients, as well the solubility of drinabant, are renorted in the table below.

| | | | |
|---|---|---|---|
| Commercial | Chemical | drinabant | Stability at RT of drinabant in the |
| name | description | solubility (mg/g) | saturated solution |

Amphiphilic surfactants with hydrophilic character (HLB > 10)

| | | | |
|---|---|---|---|
| PS80 VG | POE monooleate | 48.8 | Stable |
| Cremophor EL | POE hydrogenated | 49.4 | Stable |
| | castor oil | | |
| Solutol HS15* | PEG | 64.6 | Stable |
| | hydroxystearate | (at 40° C.) | |

Hydrophilic solvents

| | | | |
|---|---|---|---|
| Ethanol 95% | | 3.3 | ND |
| PEG 400 | Poly(ethylen | 50 | ND |
| | glycol) 400 | | |
| Propylene | | 7 | ND |
| glycol | | | |
| Glycerol | | <<0.0001 | ND |
| Glycofurol | | 139.4 | 0.15% impurity (t 19 days) |

*solid at Room Temperature (melting point 30° C.)

Taking into account the solubility results, the 3 tested surfactants were initially retained. However, Cremophor EL was discarded for further studies because of its higher toxicity compared to PS80 and to Solutol HS15. Thus the following surfactants were retained for the development of binary systems drug-surfactant:

1. PS80 (Polysorbate 80)

2. Solutol HS15

Among the tested hydrophilic solvents, the following solvents were retained for the development of ternary or quaternary systems drug-surfactant-(co)solvent(s):

1. Ethanol

2. PEG 400

3. Propylene glycol

Glycofurol was discarded, despite the very high solubility of drinabant in this solvent, because of its impurity and consequent potential toxicity unless further purification.

The weighed drug was dispersed in the excipient (surfactant or mixture of surfactant with solvents), then maintained under mechanical stirring until complete dissolution. Solutol HS15 had been previously melted in a microwave oven (resolidification of the formulation after drug dissolution). Formulations with different drug concentrations were initially prepared: 10, 25 and 40 mg/g for the binary systems, 10 and 25 mg/g for the ternary and quaternary systems.

During the screening step, the physical stability of the prototypes was evaluated as visual aspect observation after dilution with glucose 5%. Solutol-based formulations, solid at Room Temperature, required a melting step at 40° C. before dilution with glucose 5%. In the columns indicating the results (right part of the following table), the number indicates the following situations:

1. clear solution

2. turbid solution

3. particles

4. precipitate

| | | | | | | | | | | Visual aspect after | | | |
|---|---|---|---|---|---|---|---|---|---|---|---|---|---|
| | Drug | | | | | | | | | dilution with glucose 5% | | | |
| | loading | Drug concentration | | | | Surfactant concentration | | | | (under mechanical | | | |
| Surfactant | (mg/g) | (mg/g) after dilution | | | | (% v/v) after dilution | | | | stirring) | | | |
| | | 1:5 | 1:10 | 1:25 | 1:50 | 1:5 | 1:10 | 1:25 | 1:50 | 1:5 | 1:10 | 1:25 | 1:50 |
| PS80 VG | 10 | 2 | 1 | 0.4 | 0.2 | 20 | 10 | 4 | 2 | 1 | nd | nd | 1 |
| DR | 25 | 5 | 2.5 | 1 | 0.5 | | | | | 2 | 3 | 3 | 4 |
| | 40 | 8 | 4 | 1.6 | 0.8 | | | | | 4 | nd | nd | nd |
| | | 1:1 | 1:10 | 1:25 | 1:50 | 1:1 | 1:10 | 1:25 | 1:50 | 1:1 | 1:10 | 1:25 | 1:50 |
| Solutol | 10 | 5 | 1 | 0.4 | 0.2 | 50 | 10 | 4 | 2 | 1 | nd | nd | 1 |
| HS15 | 25 | 12.5 | 2.5 | 1 | 0.5 | | | | | 1 | nd | nd | 1 |
| | 40 | 20 | 4 | 1.6 | 0.8 | | | | | 1 | 1 | nd | nd |

In binary systems drug-PS80 or drug-Solutol HS15, no precipitation was observed for the 10 mg/g formulations, up to 1:50 dilution. The dilution 1:1 was not possible for PS80 since a gel was obtained (1:5 dilution chosen as the lowest dilution). At 25 mg/g, precipitation occurred immediately for the PS80 formulation (1:5 and 1:50 dilution), and only after 6 hours for the Solutol HS15 formulation. At 40 mg/g, again precipitation occurred immediately for the PS80 formulation (1:5 dilution). The physical stability of the Solutol formulation was much lower (<2 h after 1:10 dilution) than at 25 mg/g, but still better than the PS80 formulation.

Solutol HS15 was thus retained for further screening in combination with one or two (co)solvents. The solvent will have a double role: to decrease the Solutol total content in the formulation (issue of toxicity for all the surfactants after iv injection) and to maintain the formulation liquid at Room Temperature, by decreasing the melting point. No contribution of the solvent in the solubilization of drinabant is expected. On the contrary, a reduced solubilization was anticipated for mixtures with PG or Ethanol, since the solubility of drinabant in those solvents was much lower than in Solutol HS15 (<10 mg/g).

Surfactant-solvent formulations (solvent amount 10 and 50%) were loaded with drinabant (10 and 25 mg/g). As shown in the table below, a 10% solvent content allowed to maintain good solubilization properties (no main differences compared to Solutol 100% formulations). However, a solvent content of 50% led to a dramatic decrease of the drinabant solubilization. The conclusions were that 10% of solvent did not apport any real benefit: formulations were still solid at Room Temperature (not enough solvent for decreasing the Solutol melting point).

| | | | Conc. drug substance (mg/g) | | | | |
|---|---|---|---|---|---|---|---|
| | | | Before | After dilution | | Visual aspect after dilution with glucose 5% | |
| Excipient | Co-solvent | Conc. co-solvent (v/v) | dilution | 1:1 | 1:50 | 1:1 | 1:50 |
| Solutol | PEG 400 | 10 | 10 | 5 | 0.2 | =24 h | =24 h |
| HS15 | | | 25 | 12.5 | 0.5 | >5 h < 16 h | >5 h < 16 h |
| | | 50 | 10 | 5 | 0.2 | >5 h < 16 h | >5 h < 16 h |
| | | | 25 | 12.5 | 0.5 | >5 h < 16 h | >5 h < 16 h |
| | PG | 10 | 10 | 5 | 0.2 | =24 h | =24 h |
| | | | 25 | 12.5 | 0.5 | >5 h < 18 h | >5 h < 18 h |
| | | 50 | 10 | 5 | 0.2 | =24 h | =24 h |
| | | | ND | 12.5 | 0.5 | nd | nd |
| | Ethanol | 10 | 10 | 5 | 0.2 | >6 h < 18 h | =5 h |
| | | | 25 | 12.5 | 0.5 | =8 h | =3 h |
| | | 50 | 10 | 5 | 0.2 | >6 h < 18 h | =4 h |
| | | | 25 | 12.5 | 0.5 | =1 h | =1 h |

| | | | Conc. Drug | | | | |
|---|---|---|---|---|---|---|---|
| | | | substance (mg/g) | | | Visual aspect after | |
| | | | | After | | dilution with | |
| | | Conc. Co- | Before | dilution | | glucose 5% | |
| Excipient | Co-Solvent | solvent (v/v) | dilution | 1:1 | 1:50 | 1:1 | 1:50 |
| Solutol HS15 | PEG 400 | 10 | 10 | 5 | 0.2 | =24 h | =24 h |
| | | | 25 | 12.5 | 0.5 | >5 h < 16 h | >5 h < 16 h |
| | | 50 | 10 | 5 | 0.2 | >5 h < 16 h | >5 h < 16 h |
| | | | 25 | 12.5 | 0.5 | >5 h < 16 h | >5 h < 16 h |
| | PG | 10 | 10 | 5 | 0.2 | =24j | =24 h |
| | | | 25 | 12.5 | 0.5 | >5 h < 18 h | >5 h < 18 h |
| | | 50 | 10 | 5 | 0.2 | =24 h | =24 h |
| | | | nd | 12.5 | 0.5 | nd | Nd |
| | Ethanol | 10 | 10 | 5 | 0.2 | >6 h < 18 h | =5 h |
| | | | 25 | 12.5 | 0.5 | =6 h | =3 h |
| | | 50 | 10 | 5 | 0.2 | >6 h < 18 h | =4 h |
| | | | 25 | 12.5 | 0.5 | =1 h | =1 h |

After investigating prototypes loaded with drinabant, a further screening was carried out in order to identify the lowest solvent amount able to maintain the formulation at the liquid state at Room Temperature for at least 12 hours. Solutol HS15 was combined with the following solvents (10%-20%-30%-40%-50%):

1. Ethanol

2. PEG 400

3. Propylene glycol

As shown in the table below, Solutol-based formulations were still liquid with the following solvent content:
Ethanol 20% or more
Propylene glycol 30% or more

Formulations containing PEG 400 up to 50% resolidified within 3 hours. The addition of Ethanol allowed to keep liquid a Solutol-PEG 400 mixture. Indeed, the preparation of a formulation Solutol-Ethanol-PEG 400 (solvent content 15-20%, PEG 400-Ethanol ratio 1:3, 1:2, 2:1 and 3:1) showed to maintain a liquid state at least 2 days. Visual aspect of ternary systems drug-Solutol HS15-solvent after dilution with glucose 5%

| | | Co-solvent | Physical state of |
|---|---|---|---|
| Surfactant | Co-solvent | concentration (w/w) | the mixture |
| | | 10 | Liquid <2 h |
| | | 20 | ND |
| | PEG 400 | 30 | ND |
| | | 40 | ND |
| | | 50 | Liquid <3 h |
| | | 10 | Liquid <1 h |
| | | 20 | Liquid <13 h |
| Solutol HS15 | PG | 30 | Liquid <13 h |
| | | 40 | Liquid <8 days |
| | | 50 | Liquid <8 days |
| | | 10 | Liquid >8 h |
| | | | and <16 h |
| | | 20 | Liquid <8 days |
| | Ethanol | 30 | Liquid <8 days |
| | | 40 | Liquid <8 days |
| | | 50 | Liquid <8 days |

Then, in order to complete the initial part of the work, mixtures of Solutol with Ethanol (80/20) or PG (70/30) or Ethanol/PEG 400 (80/10/10) were loaded with drinabant (5-30 mg/g). For the drug loading of ≥20 mg/g, physical stability was <1 hour for any formulation after dilution with glucose 5% (dilution 1:10 or 1:20). For lower drug loadings (5-10-15 mg/g), particles appeared after 2 hours or more.

Finally, it was shown that a dilution with glucose 5% <1:10 did not assure the isotonicity of the final solution (hypertonicity).

At this step, it was decided to investigate formulations loaded at 10 mg/g, since the initially required physico-chemical shelf-life of the formulation (after addition of glucose 5%) was 6 hours. The following formulations, loaded at 10 mg/g, were identified for a complete physicochemical characterization:

1. Solutol 100%

2. Solutol 80%-Ethanol 20%

3. Solutol 70%-Propylene glycol 30%

Characterization of the final formulation (concentrate diluted 1:10 with glucose 5%) consisted of:

1. Physical stability (by visual, binocular magnifying glass and microscopy observation)

2. Chemical stability

3. Color and opalescence

4. pH

5. Osmolality

6. Injectability test

The formulation was filtered onto 0.22 μm (Durapore) immediately after dilution with glucose 5%. Indeed, previous observations showed that filtration delayed the appearance of drug crystals in the solution. In a second step, in order to increase the final drug concentration for animal studies, higher loadings (15, 20, 30, 40 mg/g) of the Solutol 100% formulation were investigated. Finally, a low drug concentration (2.7 mg/g) Solutol 100% formulation was evaluated in order to support low dose animal studies.

As shown in the following tables, Solutol-based formulations (with and without solvent) loaded at 10 mg/g exhibited a quite similar behaviour after dilution with glucose 5%. However, some rare crystals were observed in the Solutol 100% formulation at t₂₄ₕ. It has to be noted that accidentally this formulation was not filtered onto 0.22 μm immediately after dilution with glucose 5%. As reported earlier, previous observations showed that filtration immediately after dilution delayed the appearance of drug crystals in the solution. This could explain the fact that no crystals were observed with the surfactant-solvent formulations at t₂₄ₕ, despite the fact the the presence of a polar solvent should reduce the solubilization capacity of micelle formation. Since to timepoints were analysed between t₆ₕ and t₂₄ₕ, the physico-chemical stability is 6 hours for the Solutol 100%formulation, 24 h for the Solutol-Ethanol and Solutol-PG formulations.

Physico-Chemical Characteristics of Solutol Formulation (Drinabant Loading 10 mg/g, Dilution 1:10 with Glucose 5%,)

| Analysis | t₀ | t_{1 h} | t_{2 h} | t_{3 h} | t_{4 h} | t_{6 h} | t_{24 h} |
|---|---|---|---|---|---|---|---|
| HPLC (mg/g) | 1.01 | 1.04 | 1.04 | 1.04 | 1.04 | 1.04 | 1.05 |
| | 1.04 | 1.05 | 1.04 | 1.04 | 1.04 | 1.04 | 1.07 |
| Colour | ND | | | | | | Placebo: B8 |
| | | | | | | | Active: J7 |
| Opalescence (NTU, nephelometric | ND | | | | | | Placebo 2.76 |
| turbidity unit) | | | | | | | NTU |
| | | | | | | | Active: 3.25 NTU |
| Visual aspect | Clear | Clear | Clear | Clear | Clear | Rare | Rare particles |
| | | | | | | particles | |

| | | | | | |
|---|---|---|---|---|---|
| Microscopy and binocular magnifying | No | ND | No | ND | No crystals |
| glass | crystals | | crystals | | |
| pH | 5.87 | ND | 5.70 | ND | 5.66 |
| Osmolality | 448 | ND | 453 | ND | 453 |

The pH of the 3 formulations was comparable, and no evolutions were reported up to 24 h. Solutol 100% formulation is isotonic after dilution 1:10 with glucose 5%. The osmolality of the solvent-containing formulations exhibited extremely high values (*). This could be due to the presence of ethanol or PG in the formulation.

Physico-Chemical Characteristics of Solutol-Ethanol (80:20) Formulation (Drinabant Loading 10 mg/g, Dilution 1:10 with Wlucose 5%)

| Analysis | t₀ | t_{1 h} | t_{2 h} | t_{3 h} | t_{4 h} | t_{6 h} | t_{24 h} |
|---|---|---|---|---|---|---|---|
| HPLC (mg/g) | 1.02 | 1.04 | 1.04 | 1.04 | 1.05 | — | 1.08 |
| | — | 1.05 | 1.05 | 1.05 | 1.05 | 1.05 | 1.10 |
| Colour | ND | Placebo: B7 | ND | | | | Placebo: B8 |
| | | Active: B8 | | | | | Active: B8 |
| Opalescence (NTU, nephelometric | ND | Placebo: 2.80 | ND | | | | Placebo: 2.51 |
| turbidity unit) | | NTU | | | | | NTU |
| | | Active: 2.90 | | | | | Active: 2.96 |
| | | NTU | | | | | NTU |
| Visual aspect | Clear | Clear | Clear | Clear | Clear | Clear | Clear |

| | | | | | |
|---|---|---|---|---|---|
| Microscopy and binocular magnifying | No | ND | No | ND | No crystals |
| glass | crystals | | crystals | | |
| pH | 5.67 | ND | 5.63 | ND | 5.58 |
| Osmolality | 937* | ND | 934* | ND | 939* |

Physico-Chemical Characteristics of Solutol-PG (70:30) Formulation (Drinabant Loading 10 mg/g, Dilution 1:10 with Glucose 5%)

| Analysis | t₀ | t_{1 h} | t_{2 h} | t_{3 h} | t_{4 h} | t_{6 h} | t_{24 h} |
|---|---|---|---|---|---|---|---|
| HPLC (mg/g) | 1.02 | 1.03 | 1.03 | 1.02 | 1.04 | 1.03 | 1.05 |
| | 0.94 | 0.95 | 0.95 | 0.95 | 0.96 | 0.96 | 0.98 |
| Colour | ND | Placebo: B8 | ND | | | | |
| | | Active: B8 | | | | | |
| Opalescence (NTU, nephelometric | ND | Placebo: 2.39 | ND | | | | Placebo: 2.44 |
| turbidity unit) | | NTU | | | | | NTU |
| | | Active: 2.68 | | | | | Active: 2.62 |
| | | NTU | | | | | NTU |
| Visual aspect | Clear | Clear | Clear | Clear | Clear | Clear | Clear |

| | | | | | |
|---|---|---|---|---|---|
| Microscopy and binocular magnifying | No | ND | No | ND | No crystals |
| glass | crystals | | crystals | | |
| pH | 5.63 | ND | 5.52 | ND | 5.45 |
| Osmolality | 929* | ND | 929* | ND | 908* |

In order to support high dose Dobutamine Stress Echocardiogram (DSE) studies in rats (30 mg/kg), the need of increasing the final drug concentration after dilution with glucose 5% (G5%) was expressed.

In the meanwhile, after toxicity studies in rats (single dose administration of increasing concentrations of Solutol in G5%), a dilution in G5% of 1:13.33 was decided for further studies in rats (previous dilution 1:10). Indeed, a dilution of 1:13.33 leads to a final Solutol concentration of 7.5% at the administration volume of 10 mL/kg (thus to a administered dose of Solutol of 750 mg/kg). This result was consistent with toxicity data of Solutol (LD50 in rats 1000-1470 mg/kg).

As evidenced in the preceding work, Solutol 100% showed a higher solubilization capacity than in presence of a co-solvent. Thus, drug loadings up to 40 mg/g, allowing to have a final drug concentration of 3 mg/g, were investigated. Preliminary data generated during the preceding work had shown that the physical stability of such a formulation was <1 hour. A complete physico-chemical characterization confirmed that crystals appear after 30-40 min.

Physico-Chemical Characteristics of Solutol Formulation (Drinabant Loading 40 mg/g, Dilution 1:13.33 with Glucose 5%)

| Analysis | t₀ | t_{30 min} | t₁ₕ | t₂ₕ |
|---|---|---|---|---|
| HPLC (mg/g) | 3.06 | 3.03 | 3.04 | 2.95 |
| | 3.02 | 3.00 | 2.99 | 2.42 |
| | 3.03 | 3.06 | 3.07 | — |
| Colour | ND | | Active: B7, B3, JB7 | ND |
| Opalescence (NTU, | ND | | Active: 6.8. 6.5 NTU | ND |
| nephelometrc | | | | |
| turbidity unit) | | | | |
| Visual aspect | Clear | Particles in 1/3 | Paticles at t₄₀ₘᵢₙ | Precipitate |
| Microscopy and binocular | No crystals | No crystals | Crystals at t₄₀ₘᵢₙ | ND |
| magnifying glass | | | | |

The particles observed after 30-40 min after dilution with glucose 5% led to drinabant precipitation, as evidenced by the drug concentration decrease at t2h. The recommendation for this formulation is the extemporary dilution with glucose 5% just before administration.

Physico-Chemical Characteristics of Solutol Formulation (Drinabant Loading 2.7 mg/g, Dilution 1:13.33 with Glucose 5%)

| Analysis | t₀ | t₁ₕ | t₈ₕ | t₂₄ₕ |
|---|---|---|---|---|
| HPLC(m/g) | 0.178 | 0.178 | 0.179 | 0.180 |
| | 0.204 | 0.205 | 0.205 | 0.209 |
| Colour | ND | Active: JB7, JB7 | Active: B8, B8 | Active: B8. JB7 |
| Opalescence (NTU, | ND | 2.56 NTU, | 2.50 NTU, | 2.50 NTU, |
| nephelometric turbidity unit) | | 2.61 NTU | 2.60 NTU | 2.57 NTU |
| Visual aspect | Clear | Clear | Clear | Clear |

No issues were reported for the 2.7 mg/g formulation (concentration after dilution 0.2 mg/g), confirming, as expected, a physico-chemical stability of 24 hours at least.

The following formulations, loaded at 10 mg/g, were diluted 1:10 with glucose 5% (final drug concentration 1 mg/mL):

1. Solutol 100%

2. Solutol 80%-Ethanol 20%

3. Solutol 70%-Propylene glycol 30%

Conditions simulated, in terms of formulation-buffer ratio, the rat model. An attempt of using a dynamic test, in order to simulate an infusion in rats at 1 mL/min, failed because of the higher viscosity of the formulation compared to the Sorensen buffer. No precipitation was observed for the 3 formulations. Thus, the test was performed with a Solutol 100% formulation with higher drug loadings:

1. 30 mg/g, diluted 1:10 for a final drug concentration of 3 mg/mL

2. 40 mg/g, diluted 1:13.33 for a final drug concentration of 3 mg/mL

Again, no precipitation was observed, suggesting that drug should not precipitate after intravenous injection for all the tested formulations.
Chemical Stability Data of Solutol and Solutol-Cosolvent Formulations (Drinabant Loading 10 mg/g)

| | Bulk Solutol HS15 | | Bulk Solutol HS15 80%- | | Bulk Solutol HS15 | |
|---|---|---|---|---|---|---|
| | 100% | | Ethanol 20% | | 70%-PG 30% | |
| | (GG 63.24A) | | (GG 63.13B) | | (GG 63.24C) | |
| | Assay | Impurities | Assay | Impurities | Assay | Impurities |
| | (mg/g) | (% w/w) | (mg/g) | (% w/w) | (mg/g) | (% w/w) |
| t₀ | 9.83 | Not detected | 9.78 | Not detected | 9.81 | Not detected |
| Temperature | t_{1 week} | | t_{1 week} | | t_{1 week} | |
| 25° C./60% RH | 9.95 | Not detected | 10.22 | Not detected | 9.88 | Not detected |
| 60° C. | 8.94 | 2.4 | 10.06 | Not detected | 9.96 | Not detected |

As shown in the table above, the 3 Solutol-based formulations, with or without solvent, when loaded at 10 mg/g with drinabant exhibit a good stability at one week at Room Temperature. However, at 60° C., a degradation impurity appeared for the Solutol 100% formulation only. Since 60° C. is an aggressive condition, it was decided to switch to the 40° C. temperature (for accelerated conditions) for the next studies.

As shown in the table below, Solutol 100% formulations loaded up to 40 mg/g exhibited a slight increase of the degradation impurity at both 25° C./60% RH (0.15%-0.3%) and 40° C./75% RH (up to 1.2%). Interestingly, the highest impurity content was detected with the lowest bulk concentration.

Chemical Stability Data of Solutol Bulk Formulation (Drinabant Loading 2.7 mg/g, 10 mg/g and 40 mg/g)

| | Bulk 2.7 mg/g | | | |
|---|---|---|---|---|
| | Assay (mg/g) | | Impurities (% w/w) | |
| t₀ | 2.74 | | Not detected | |
| Temperature | 1 week | 3 weeks | 1 week | 3 weeks |
| 5° C. | ND | 2.72 | ND | <0.1 |
| 25° C./60% RH | 2.77 | 2.72 | 0.15 | 0.3 |
| 40° C./75% RH | 2.48 | 2.72 | 0.65 | 1.2 |
| | Bulk 10 mg/g | | | |
| | Assay (mg/g) | | Impurities (% w/w) | |
| t₀ | 9.83 | | Not detected | |
| Temperature | 1 week | 3 weeks | 1 week | 3 weeks |
| 5° C. | ND | | | |
| 25° C./60% RH | 9.95 | ND | — | ND |
| 40° C./75% RH | ND | | | |
| | Bulk 40 mg/g | | | |
| | Assay (mg/g) | | Impurities (% w/w) | |
| t₀ | 41.4 | | Not detected | |
| Temperature | 1 week | 3 weeks | 1 week | 3 weeks |
| 5° C. | ND | 39.1 | ND | <0.1 |
| 25° C./60% RH | 39.4 | 40 | 0.17 | 0.21 |
| 40° C./75% RH | 39.7 | 39.9 | 0.5 | 0.66 |

A Solutol-based solution for iv administration has been successfully developed. For PK and DSE studies, a Solutol concentrate is diluted in glucose 5% solution before administration and exhibits the following physico-chemical stability depending on drinabant loading:
1.5 mg/mL after dilution with G5% (Bulk 15 mg/g): 24 hours at RT
2 mg/mL after dilution with G5% (Bulk 20 mg/g): 6 hours RT
3 mg/mL after dilution with G5% (Bulk 30 mg/g): 2 hours at RT
3 mg/mL after dilution with G5% (Bulk 40 mg/g): 20 min at RT

This formulation has been used for the following iv studies:
PK in dogs (dose 0.5 mg/kg, final concentration 0.2525 mg/mL, bulk at 20 mg/g)
PK in rats (dose 5 mg/kg, final concentration 0.5 mg/mL, bulk at 6.665 mg/g)
Single dose toxicity study in rats (dose up to 30 mg/kg, final concentration 3 mg/mL, bulk at 40 mg/g)

For human administration, the concentrate was composed of Solutol-Ethanol 80:20 in order to avoid a melting step before dilution in the infusion bag.

Although the present invention has been described with reference to specific details of certain embodiments thereof in the above examples, it will be understood that modifications and variations are encompassed within the spirit and scope of the invention.

What is claimed is:

1. A parenteral fluid concentrate comprising:
an amount of drinabant or a salt or polymorph thereof effective to i) treat, reverse, or reduce acute cannabinoid overdose or one or more symptoms thereof in a subject in need thereof, and/or ii) treat, reverse, or reduce cannabinoid hyperemesis syndrome (CHS) or one or more symptoms thereof in a subject in need thereof; and
at least one agent that acts as a non-ionic solubilizer and/or emulsifying agent.

2. The concentrate of claim 1, wherein the at least one agent that acts as a non-ionic solubilizer and/or emulsifying agent is chosen from polyoxyethylene (20) sorbitan monooleate, polyoxyl 40 hydrogenated castor oil, polysorbate 80; macrogol 15 hydroxystearate, and polyoxamers.

3. The concentrate of claim 2, wherein the at least one agent that acts as a non-ionic solubilizer and/or emulsifying agent is chosen from polysorbate 80 and rnacrogol 15 hydroxystearate.

4. The concentrate of claim 3, wherein the at least one agent that acts as a non-ionic solubilizer and/or emulsifying agent is macrogol 15 hydroxystearate.

5. The concentrate of any one of the preceding claims, wherein the concentrate further comprises at least one hydrophilic solvent.

6. The concentrate of claim wherein the at least one hydrophilic solvent is chosen from ethanol, PEG 400, and propylene glycol.

7. The concentrate of any one of claims 1 to 6, wherein the concentrate consists of an amount of drinabant or a salt or polymorph thereof effective to reverse acute cannabinoid overdose or one or more symptoms thereof or reverse cannabinoid hyperemesis syndrome or one or more symptoms thereof in a subject and macrogol 15 hydroxystearate.

8. The concentrate of any one of claims 1 to 6, wherein the concentrate consists of an amount of drinabant or a salt or polymorph thereof effective to reverse acute cannabinoid overdose, or one or more symptoms thereof in a subject, and a mixture of 80% macrogol 15 hydroxystearate, and 20% ethanol.

9. The concentrate of any one of claims 1 to 6, wherein the concentrate consists of an amount of drinabant or a salt or polymorph thereof effective to reverse acute cannabinoid overdose, or one or more symptoms thereof in a subject, and a mixture of 70% macrogol 15 hydroxystearate, and 30% propylene glycol.

10. The concentrate of any one of the preceding claims, wherein the concentrate comprises between about 2 and about 50 mg/g with drinabant or a salt or polymorph thereof.

11. The concentrate of claim 10, wherein the concentrate comprises 10 mg/g drinabant or a salt or polymorph thereof.

12. The concentrate of any one of the preceding claims, wherein the concentrate is stable for one week at room temperature.

13. The concentrate of any one of the preceding claims, wherein the composition further comprises at least one phospholipid.

14. The concentrate of claim 13, wherein the at least one phospholipid is chosen from lecithins of natural origin, phospholipids of natural origin, synthetic phospholipids, and a mixture thereof.

15. The concentrate of any one of the preceding claims, wherein the composition further comprises Miglyol 812, a pharmaceutical oil, or a mixture thereof.

16. The concentrate of claim 15, wherein the pharmaceutical oil is chosen from soybean oil, olive oil, sesame oil, and hydrogenated vegetable oil.

17. The concentrate of any one of the preceding claims, wherein the composition further comprises at least one antioxidant.

18. The concentrate of claim 17, wherein the at least one antioxidants chosen from ascorbic acid, monothioglycerol, cysteine HCl, and glutathione.

19. The concentrate of any one of the preceding claims, wherein the composition further comprises at least one isotonic agent.

20. The concentrate of claim 19, wherein the at east one isotonic agent is chosen from polyethylene glycol, glycerol, saline, and glucose.

21. A parenteral fluid comprising a concentrate of any one of the preceding claims diluted with a pharmaceutically acceptable aqueous carrier.

22. The fluid of claim 21, wherein the pharmaceutically acceptable aqueous carrier is glucose 5% solution.

23. The fluid of claim 21 or 22, wherein the fluid comprises 1.5 mg/mL drinabant or a salt or polymorph thereof.

24. The fluid of claim 21 or 22, wherein the fluid comprises 2 mg/mL drinabant or a salt or polymorph thereof.

25. The fluid of claim 21 or 22, wherein the fluid comprises 3 mg/mL drinabant or a salt or polymorph thereof.

26. A method of treating, reversing, or reducing one or more symptoms of acute cannabinoid overdose comprising parenterally administering the parenteral fluid of any one of claims 21 to 25 to a subject in need thereof.

27. The method of claim 26, wherein the symptom(s) of acute cannabinoid overdose is/are chosen from cardiovascular symptom(s), neuropsychiatric symptom(s), and gastrointestinal symptom(s).

28. The method of claim 27, wherein the cardiovascular symptom(s) is/are chosen from hypertension and tachycardia.

29. The method of claim 27, wherein the neuropsychiatric symptom(s) is/are chosen from agitation, confusion, drowsiness/lack of alertness, hallucinations, and feeling “high.”

30. The method or of claim 27, wherein the gastrointestinal symptom(s) is/are chosen from nausea and vomiting.

31. The method of any one of claims 26 to 30, wherein the onset of reversal of symptom(s) of acute cannabinoid overdose are apparent within 5-30 minutes following intravenous injection of drinabant, or a salt or polymorph thereof.

32. The method of any one of claims 26 to 31, wherein the onset of reversal of symptom(s) of acute cannabinoid overdose are apparent within 15-45 min following intramuscular administration of drinabant, or a salt or polymorph thereof.

33. The method of any one of claims 26 to 32, wherein if no response is observed within 30-120 minutes of a first administration of drinabant, and the presence of cannabinoids is confirmed or strongly suspected, a second dose may be administered.

34. The method of any one of claims 26 to 33, wherein if no response is observed within 30-45 minutes of a first administration of drinabant, and the presence of cannabinoids is confirmed or strongly suspected, a second dose may be administered.

35. A method of treating, reversing, or reducing cannabinoid hyperemesis syndrome or one or more symptoms thereof comprising parenterally administering the parenteral fluid of any one of claims 21 to 25 to a subject in need thereof.

36. The method of any one of claims 26 to 35, wherein the parenteral route of administration is chosen from among intravenous (IV), intramuscular (IM), and subcutaneous (SC).

37. The method of claim 36, wherein the parenteral route of administration is IV.

38. The method of claim 37, wherein the amount of drinabant, or a salt or polymorph thereof, is between about 1 mg and about 60 mg per intravenous dose.

39. The method of claim 38, wherein the amount of drinabant, or a salt or polymorph thereof is between about 30 and about 60 mg per intravenous dose.

40. The method of claim 36, wherein the IV dose is delivered by IV injection.

41. The method of claim 40, wherein the IV dose is delivered in a liquid volume of between about 1 and about 20 mL.

42. The method of claim 36, wherein the IV dose is delivered by IV infusion.

43. The method of claim 40, wherein the IV infusion is delivered in a liquid volume of between about 125 to about 500 mL.

44. The method of claim 42 or 43, wherein the IV infusion is delivered over a period of about 1 hour to about 2 hours.

45. The method of any one of claims 42 to 44, wherein the IV infusion is delivered at a rate of about 0.5 mL/min to about 2 mL/min.

46. The method of claim 36, wherein the parenteral route of administration is IM or SC.

47. The method of claim 46, wherein the amount of drinabant, or a salt or polymorph thereof is between about 5 and about 60 mg per IM or SC dose.

48. The method of claim 47, wherein the amount of drinabant, or a salt or polymorph thereof is between about 5 and about 30 mg per IM or SC dose.

49. The method of any one of claims 45 to 48, wherein the intramuscular dose is delivered in a liquid volume of up to about 2.5 ml.

50. The method of claim 49, wherein the intramuscular dose is delivered in a liquid volume of about 1 to about 2.5 ml.

51. The method of claim 48, wherein the SC dose is delivered in a liquid volume of up to about 1.5 ml.

52. The method of claim 46 or 47, wherein the SC dose is delivered in a liquid volume of about 1 mL to about 1.5 ml.
